(19) United States
(12) Patent Application Publication
(10) Pub. No.: US 2010/0286543 A1
(43) Pub. Date: Nov. 11, 2010

(54) Automated Cardiac Status Determination System

(75) Inventors: Ravindra Balasaheb Patil, Bangalore (IN); Preetham Nagaraja Murthy, Bangalore (IN)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC., Malvern, PA (US)

(21) Appl. No.: 12/750,900

(22) Filed: Mar. 31, 2010

Related U.S. Application Data
(60) Provisional application No. 61/175,627, filed on May 5, 2009.

(51) Int. Cl. A61B 5/0452

(52) U.S. Cl. 600/523

(57) ABSTRACT

A system fits a curve to a filtered ECG signal, processes the fitted curve (e.g., by applying transforms such as a KL Transform) and derives parameters for use in classifying heart cycle signal portions (such as an ST segment portion) into particular heart cycle signal portion categories associated with particular segment morphology. A system for heart signal classification includes an interface for receiving an electrical signal waveform comprising an R-wave and including an ST segment portion associated with heart electrical activity of a patient over a heart beat cycle. A signal processor processes data representing the electrical signal waveform by fitting a curve to the ST segment and applying a transform to the fitted curve to derive variance data indicating variance in the fitted curve. A signal classifier classifies the ST segment into one of multiple predetermined categories associated with the fitted ST segment curve geometry in response to the derived variance data.

This is a non-provisional application of provisional application serial No. 61/175,627 filed May 5, 2009, by P. N. Murthy et al.

### FIELD OF THE INVENTION

This invention concerns a system for heart signal classification by classifying a heart signal in a portion of a heart cycle into one of multiple predetermined categories in response to determined signal voltage difference and variances.

### BACKGROUND OF THE INVENTION

An Electrocardiogram (ECG) is used by cardiologists to aid in the diagnosis of various cardiac abnormalities. Cardiac arrhythmia and ischemia are some of the conditions that are identified through the analysis of an ECG. The morphology of an ST segment is an important clinical parameter in identifying a type of heart attack. Some of these types are ST Elevation Myocardial Infarction (STEMI) and Non ST Elevation Myocardial Infarction (NSTEMI) which can be identified through ST segment morphology. Further, the shape and geometry of the ST morphology is also used as an indicator of an impending heart attack and to identify severity of a heart attack. FIG. 1 shows a single ECG heart cycle showing fiducial points and segments including the ST segment.

Known cardiac status determination systems involve the use of slope determination, and Karhunen-Loève (KL) Transforms on a raw signal to detect ischemic events, for example. However known systems are limited and lack a comprehensive capability to identify cardiac status. A system according to invention principles addresses these deficiencies and related problems.

### SUMMARY OF THE INVENTION

A system automatically fits a curve to a filtered ECG signal, processes the fitted curve (e.g., by applying transforms such as a KL Transform) and automatically derives parameters (e.g., a ΔJTon parameter) for use in classifying heart cycle signal portions (such as an ST segment portion) into particular heart cycle signal portion categories associated with particular segment morphology (such as Horizontal Depression and Downsloping Depression, for example). A system for heart signal classification includes an interface for receiving an electrical signal waveform comprising an R-wave and including an ST segment portion associated with heart electrical activity of a patient over a heart beat cycle. A signal processor processes data representing the electrical signal waveform by fitting a curve to the ST segment and applying a transform to the fitted curve to derive variance data indicating variance in the fitted curve. A signal classifier classifies the ST segment into one of multiple predetermined categories associated with the fitted ST segment curve geometry in response to the derived variance data.

### BRIEF DESCRIPTION OF THE DRAWING

FIG. 1 shows fiducial points and segments of an ECG signal indicating heart electrical activity over a heart cycle.

FIG. 2 shows a system for heart signal classification, according to invention principles.

FIG. 3 shows a flowchart of a process for categorizing ST Segment Morphology into classes, according to invention principles.

FIG. 4 shows an ECG signal indicating heart electrical activity over a heart cycle showing a ΔJTₒₙ characteristic that facilitates differentiation between classes including Horizontal Depression and Downsloping Depression, according to invention principles.

FIG. 5 shows an ECG signal indicating heart electrical activity over a heart cycle showing a ΔJTₒₙ characteristic comprising a Downsloping Depression, according to invention principles.

FIG. 6 shows an ST Segment comprising a Convex Elevation, according to invention principles.

FIG. 7 shows a curve fit to an ST segment portion of the Convex Elevation of FIG. 6, according to invention principles.

FIG. 8 shows a curve fit for an ST Segment showing a Concave Elevation, according to invention principles.

FIG. 9 shows a curve fit for an ST Segment showing an Upsloping Depression, according to invention principles.

FIG. 10 shows a curve fit for an ST Segment showing a Horizontal Depression, according to invention principles.

FIG. 11 shows a flowchart of a process for categorizing ST Segment Morphology into classes in response to fitting a curve to the segment, according to invention principles.

FIG. 12 shows a flowchart of a process used by a system for heart signal classification, according to invention principles.

FIG. 13 shows characteristics of an ST segment used by a signal classifier to classify the ST segment, according to invention principles.

### DETAILED DESCRIPTION OF THE INVENTION

A system fits a curve to a filtered ECG signal, processes the fitted curve (e.g., by applying transforms such as a KL Transform) and derives parameters (e.g., a ΔJTon parameter) for use in classifying heart cycle signal portions. Specifically, the system comprises an automated ST Morphology classifier that classifies an ST segment portion into particular heart cycle signal portion categories including Concave Elevation, Convex Elevation, Upsloping Depression, Horizontal Depression and Downsloping Depression, for example, associated with particular segment morphology.

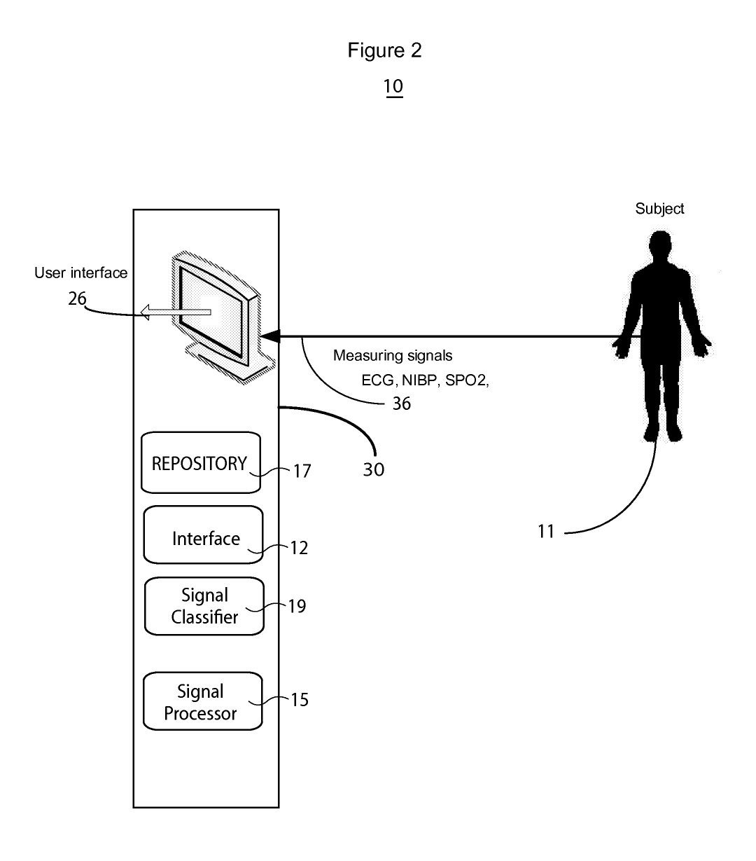

FIG. 2 shows system 10 for heart signal classification. System 10 comprises at least one processing device 30 comprising a server, computer, notebook, PDA, phone or other device including a user interface 26, interface 12, signal processor 15, signal classifier 19 and at least one repository 17. Interface 12 receives an electrical signal waveform 36 (e.g., an ECG waveform) derived from patient 11 comprising an R-wave and including an ST segment portion associated with heart electrical activity of a patient over a heart beat cycle. Signal processor 15 processes data representing the electrical signal waveform by fitting a curve to the ST segment and applying a transform to the fitted curve to derive variance data indicating variance in the fitted curve. Signal processor 15 processes data representing the electrical signal waveform by identifying a J point in the electrical signal waveform and identifying a Ton point in the electrical signal waveform substantially occurring 80 milliseconds after the J point and also determining a voltage difference between J point and Ton electrical signal waveform values. Signal classifier 19 classifies the ST segment into one of multiple predetermined categories in response to the derived voltage difference value and also classifies the ST segment into one of multiple predetermined categories associated with the fitted ST segment curve geometry in response to the derived variance data. Resultant classification data and associated electrical signal waveform 36 are stored together with other patient medical parameters and demographic (age, gender, height, weight) data in at least one repository 17. User interface 26 presents at least one display image indicating ST segment category data and presenting an electrical signal waveform including an identified J point and Ton value.

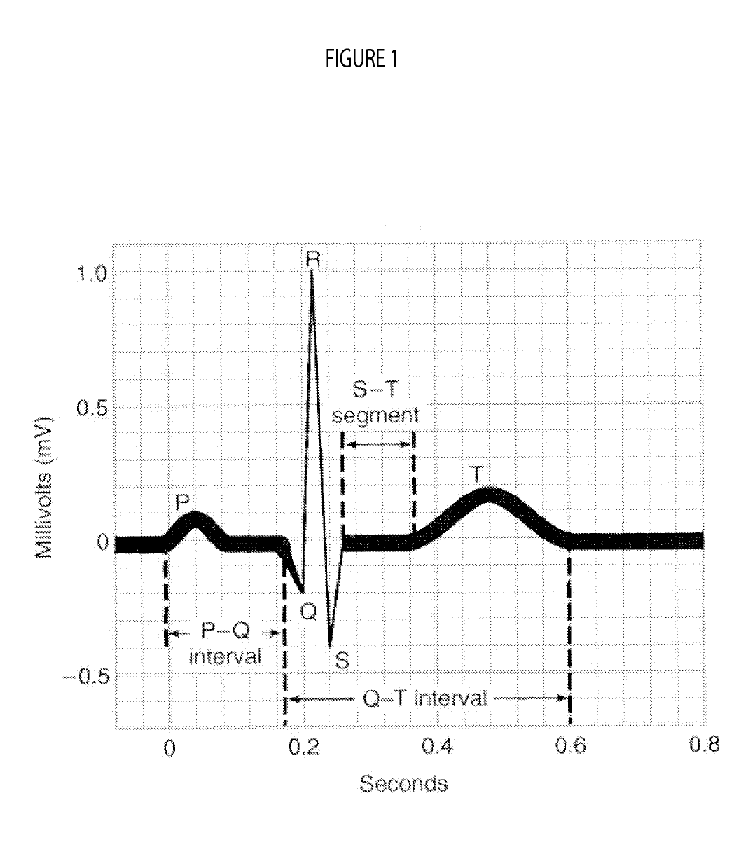

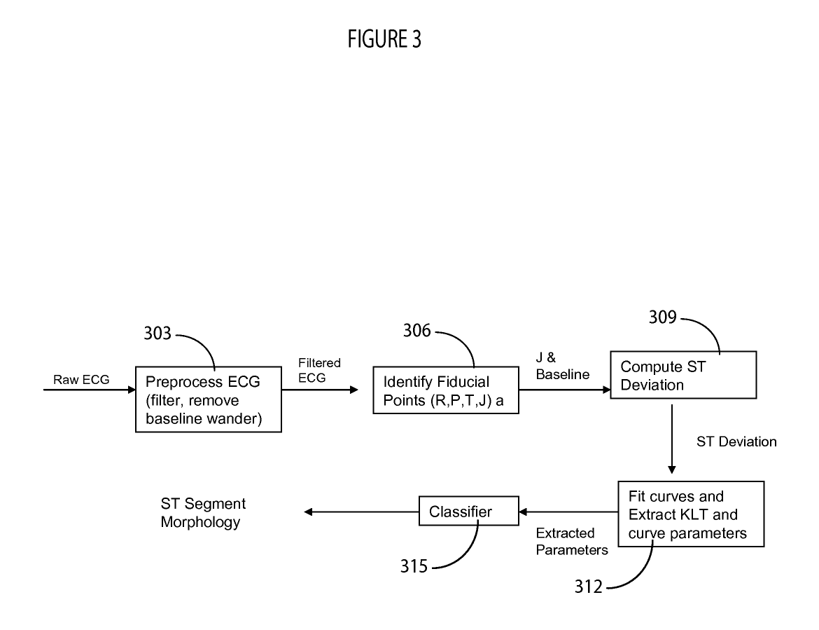

FIG. 3 shows a flowchart of a process for categorizing ST Segment Morphology into classes performed by system 10 (FIG. 2). Signal processor 15 (FIG. 1) in step 303 preprocesses data representing electrical signal (e.g., ECG) waveform 36 derived from patient 11 by filtering and removing baseline drift. Processor 15 in step 306 identifies Fiducial points (including R, P, T, J points) in the preprocessed electrical signal waveform. In step 309, signal processor 15 processes data representing the preprocessed electrical signal waveform to compute an ST segment deviation (indicating ST segment slope is positive or negative). Signal processor 15 in step 312 further processes data representing the electrical signal waveform by fitting a curve to the ST segment and applying a Karhunen-Loève Transform (KLT), for example, to the fitted curve and extracting KLT parameters for deriving variance data indicating variance in the fitted curve from a corresponding curve for a patient having substantially matching demographic characteristics (age, weight, height, gender, pregnancy, for example). Signal classifier 19 in step 315 classifies the ST segment into one of multiple predetermined categories associated with the fitted ST segment curve geometry in response to the derived variance data. The associated categories including Concave Elevation, Convex Elevation, Upsloping Depression, Horizontal Depression and Downsloping Depression, for example, associated with particular segment morphology.

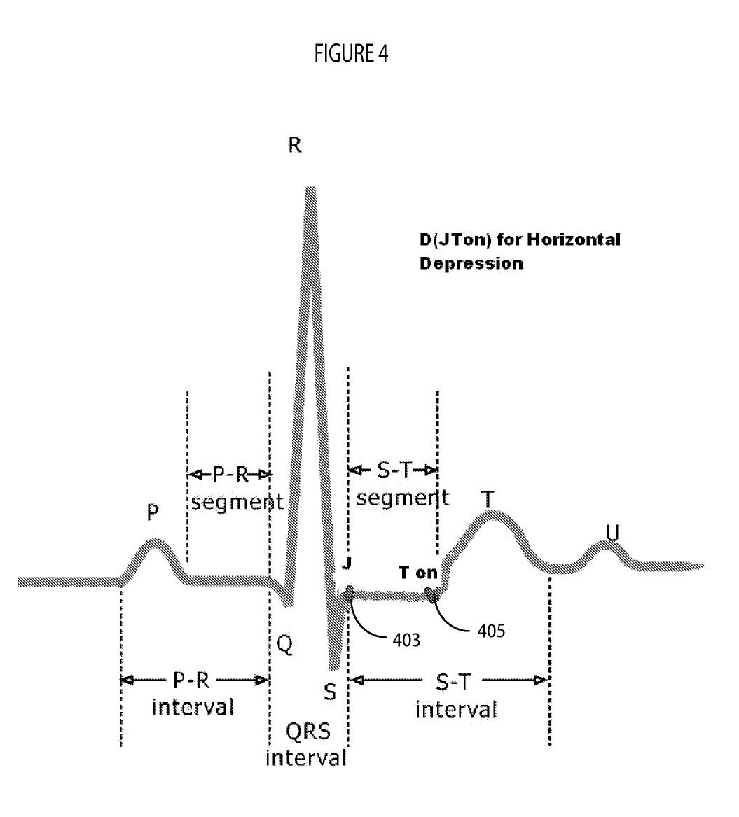

System 10 extracts parameters using a KL transform, for example, applied to a curve fitted to an ST segment and identifies ΔJTₒₙ. This facilitates differentiating between ST segment classes including Horizontal Depression and Downsloping Depression. FIG. 4 shows an ECG signal indicating heart electrical activity over a heart cycle showing a ΔJTₒₙ characteristic and illustrating an ST segment horizontal depression. The ΔJTₒₙ characteristic facilitates differentiation between classes including Horizontal Depression and Downsloping Depression and comprises a voltage difference in the waveform between voltages at an ST segment start point 403 (a J point) and the onset of a T wave (Ton) point 405 empirically taken to occur 80 milliseconds after the J point.

ΔJTₒₙ=ECG (Ton)−ECG (J).

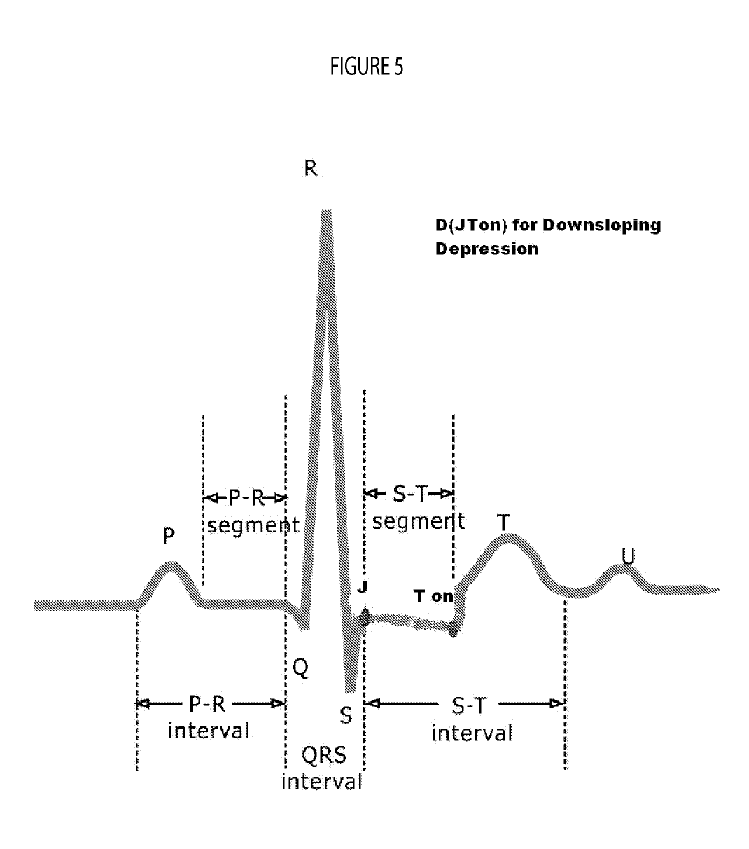

Similarly, FIG. 5 shows an ECG signal showing a ΔJTₒₙ characteristic comprising a Downsloping Depression.

In one embodiment, system 10 applies a known Karhunen Loeve Transform (KLT) to a curve fitted to an ST segment. The Karhunen Loeve Transform is also known as Principal Component Analysis and is mathematically defined as an orthogonal linear transformation that transforms data to a new coordinate system such that the greatest variance by any projection of the data comes to lie on the first coordinate (called the first principal component), the second greatest variance on the second coordinate, and so on. KLT is theoretically the optimum transform for given data in least square terms.

Principal component analysis (PCA) involves a mathematical procedure that transforms a number of possibly correlated variables into a smaller number of uncorrelated variables called principal components. The first principal component accounts for as much of the variability in the data as possible, and each succeeding component accounts for as much of the remaining variability as possible. Depending on the field of application, it is also named the discrete Karhunen-Loève transform (KLT). PCA operation can be thought of as revealing the internal structure of data in a way which best explains the variance in the data. If a multivariate dataset is visualised as a set of coordinates in a high-dimensional data space (1 axis per variable), PCA supplies the user with a lower-dimensional picture, a “shadow” of this object when viewed from its (in some sense) most informative viewpoint. The low-order principal components often contain the most important aspects of the data. However, depending on the application this may not always be the case.

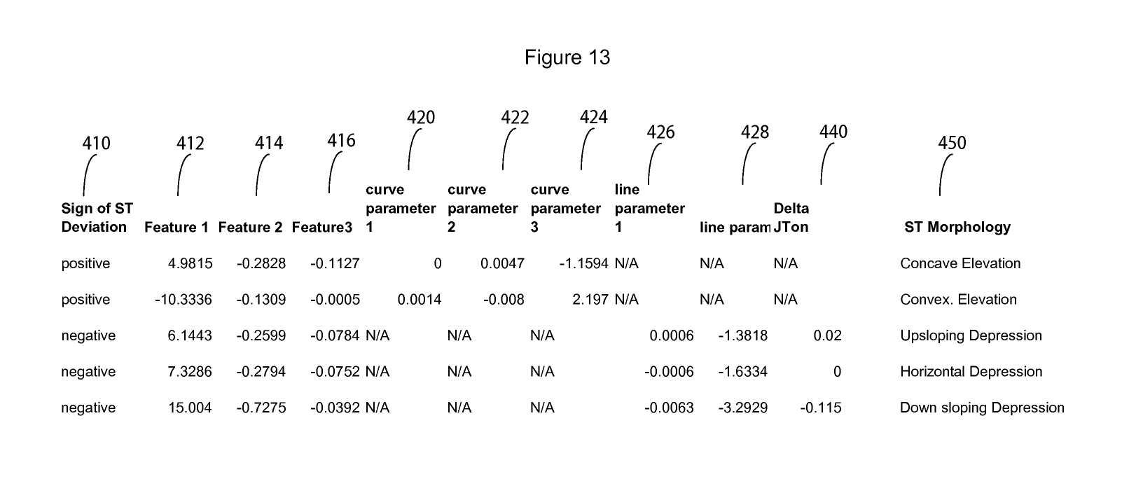

FIG. 13 shows characteristics of an ST segment derived by signal processor 15 (FIG. 2) that are used by signal classifier 19 to classify an ST segment. The ST segment characteristics include a sign of an ST segment deviation (e.g., positive or negative) in column 410 and PCA components in columns 412, 414 and 416 (Features 1, 2 and 3) derived by applying a (KLT) transform to a curve fitted to the ST segment. The ST segment characteristics further include Curve parameters 1, 2 and 3 in columns 420, 422 and 424 respectively and line parameters in columns 426 and 428 that are the parameters of the fitted curve or line. Signal processor 15 determines ST Deviation and fits a curve to the ST segment if the ST segment deviation is positive and fits a line to the ST segment if the ST segment deviation is negative. Signal processor 15 further determines ΔJTₒₙ shown in column 440 as previously explained. Signal classifier 19 categorizes an ST segment as indicated in column 450 into categories including Concave Elevation, Convex Elevation, Upsloping Depression, Horizontal Depression and Downsloping Depression, for example, associated with particular segment morphology.

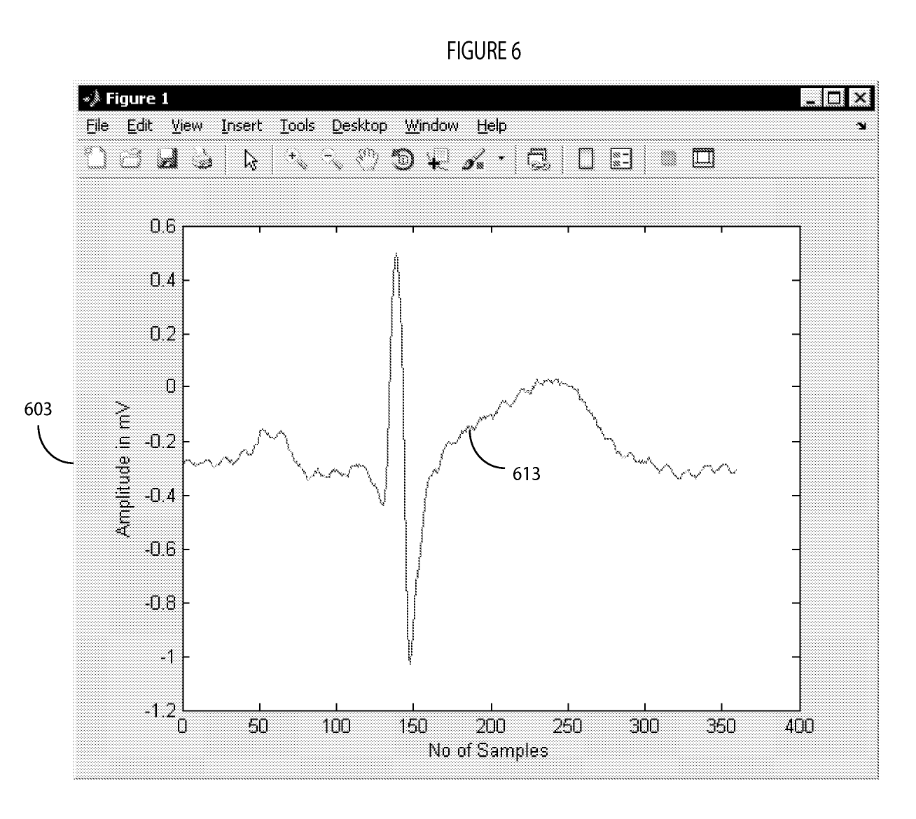

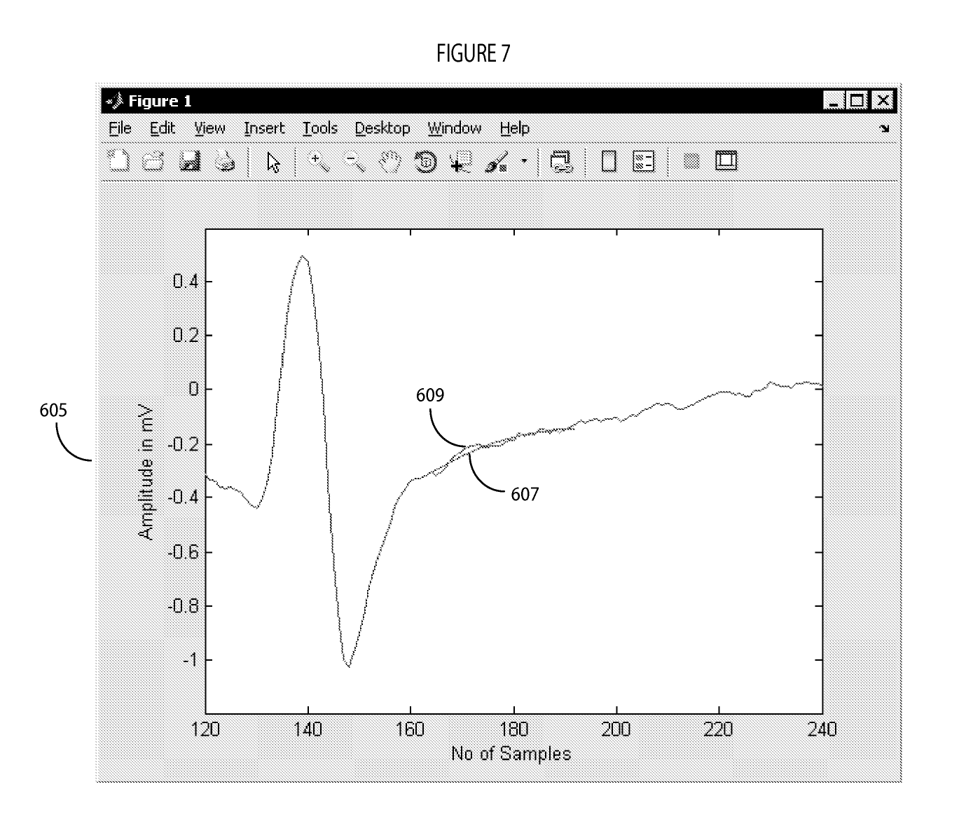

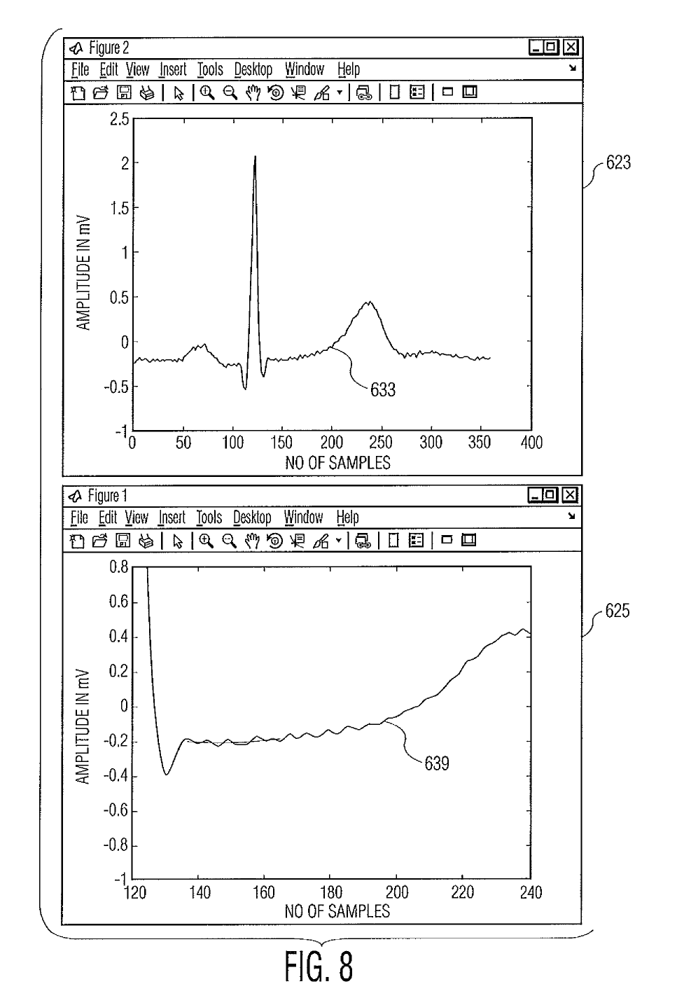

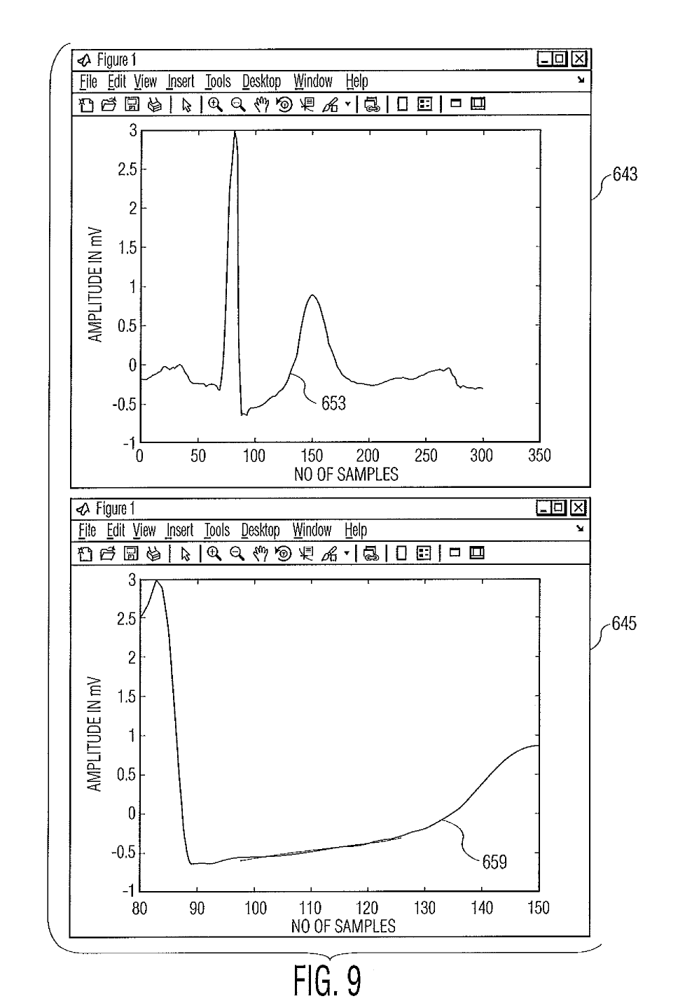

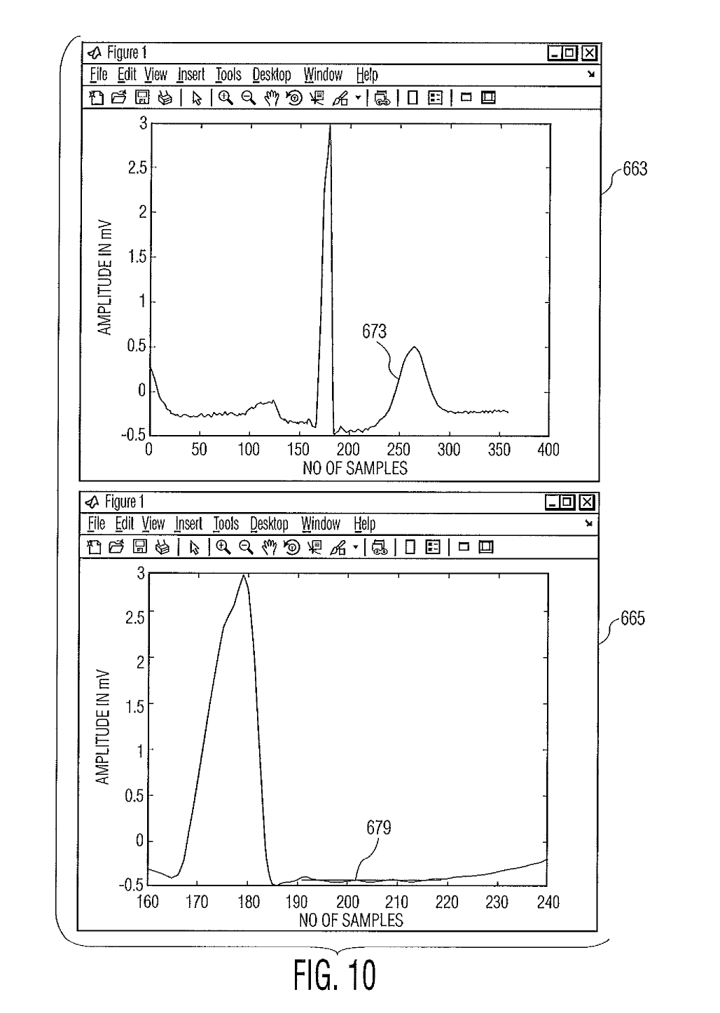

FIG. 6 image 603 shows an electrical signal waveform of heart electrical activity including ST Segment 613 comprising a Convex Elevation. FIG. 7 image 605 shows a curve fit 607 provided by signal processor 15 (FIG. 2) to an expanded portion 609 of the ST segment 613 of FIG. 6. FIG. 8 image 623 shows an electrical signal waveform of heart electrical activity including ST Segment 633 comprising a Concave Elevation. Image 625 shows a curve fit provided by signal processor 15 (FIG. 2) to an expanded portion 639 of the ST segment 633. FIG. 9 image 643 shows an electrical signal waveform of heart electrical activity including ST Segment 653 comprising an Upsloping Depression. Image 645 shows a curve fit provided by signal processor 15 (FIG. 2) to an expanded portion 659 of the ST segment 653. FIG. 10 image 663 shows an electrical signal waveform of heart electrical activity including ST Segment 673 comprising a Horizontal Depression. Image 665 shows a curve fit provided by signal processor 15 (FIG. 2) to an expanded portion 679 of the ST segment 673.

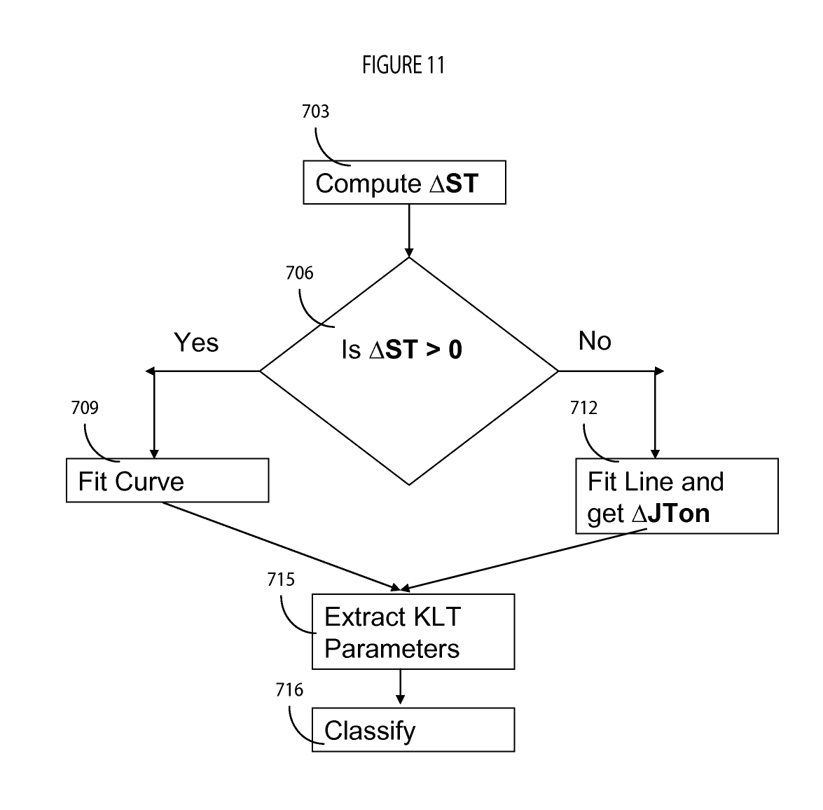

FIG. 11 shows a flowchart of a process for categorizing ST Segment Morphology into classes in response to fitting a curve to an ST segment. Signal processor 15 in step 703 determines an ST Deviation value (indicating ST segment slope is positive or negative) and in step 706 determines to fit either a second degree curve or a first degree curve (line) to the ST Segment. For ST elevation (i.e., positive ST Deviation) signal processor 15 in step 709 fits a second degree curve because the morphology class (Concave or Convex Elevation) is better identified through a second degree curve. For ST depression (i.e., negative ST Deviation), signal processor 15 in step 712 fits a line as the morphology class (upsloping, downsloping, or horizontal) is better identified through a line. Signal processor 15 in step 715 advantageously applies a KL Transform over a curve fitted segment (in contrast to applying a KL Transform to a raw ST segment. Signal processor 15 further derives a parameter ΔJTₒₙ and uses the parameter to improve resolution between classes Horizontal Depression and Downsloping Depression. Signal processor 15 extracts and employs KLT features from a curve or line fit of an ECG signal segment (the ST segment) comprising curve and line parameters as indicated in FIG. 13 advantageously including a ΔJTon parameter. Signal classifier 19 in step 716 uses the parameters to improve classification of ST segment morphology (Horizontal Depression and Downsloping Depression, for example) into specific classes.

The presence of noise in an electrical signal indicating heart activity, exacerbates the difficulty of identifying morphology of the signal. ECG signals are prone to noise which distorts the signal. This distortion affects the successful morphological classification of the signal. Hence signal processor 15 filters an ECG signal to remove noise and advantageously automatically fits a curve to address this problem as the curve fit captures the geometry of an ST segment. System 10 in one embodiment captures extracted signal parameters including KLT parameters, which facilitate data compression. The difference between the class Downsloping Depression and Horizontal Depression is difficult to resolve even with KLT and curve parameters. Hence system 10 uses the ST Deviation value to determine the degree to which the segment is horizontal or downsloping which provides higher accuracy in differentiating between these two classes.

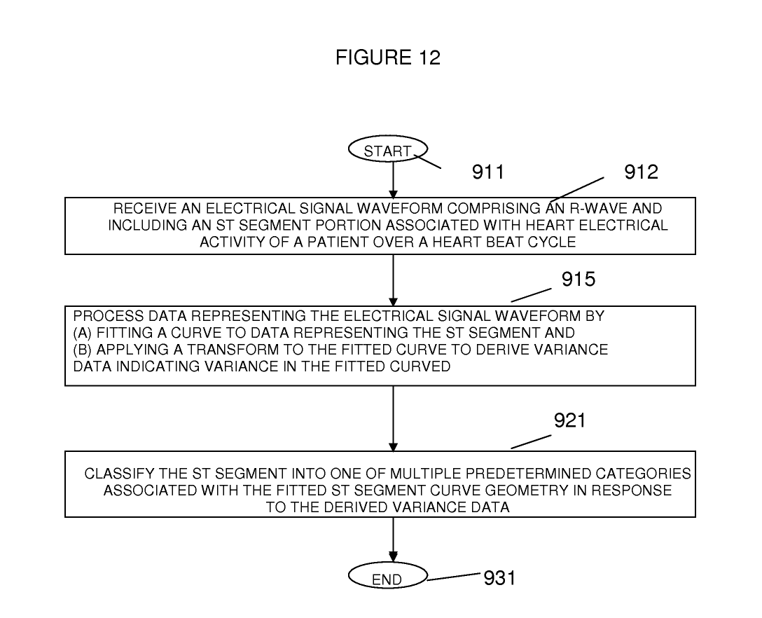

FIG. 12 shows a flowchart of a process used by system 10 for heart signal classification. In step 912 following the start at step 911, system 10 receives an electrical signal waveform comprising an R-wave and including an ST segment portion associated with heart electrical activity of a patient over a heart beat cycle. Signal processor 15 in step 915 processes data representing the electrical signal waveform by (a) fitting a curve to data representing the ST segment and (b) applying a transform to the fitted curve to derive variance data indicating variance in the fitted curve. Signal processor 15 adaptively fits a first degree curve or a second degree curve selected in response to a determined ST deviation value indicating a positive or negative ST segment slope. Signal processor 15 further adaptively fits a curve or a line to an ST segment, selected in response to the determined ST deviation value. Also processor 15 processes data representing the electrical signal waveform by, identifying a J point in the electrical signal waveform, identifying a Ton point in the electrical signal waveform substantially occurring 80 milliseconds after the J point and determining a voltage difference between J point and Ton electrical signal waveform values. The transform comprises a KLT transform or another variance analysis transform. The KLT transform performs Principal Component Analysis (PCA) to transform the data to a new coordinate system such that the greatest variance lies on a first coordinate called the first principal component.

In step 921 signal classifier 19 classifies the ST segment into one of multiple predetermined categories associated with the fitted ST segment curve geometry in response to the derived variance data and in response to the derived voltage difference value. Specifically, signal classifier 19 classifies the ST segment into one of multiple predetermined categories associated with characteristics including, Concave Elevation, Convex Elevation, Upsloping Depression, Horizontal Depression and Downsloping Depression. Signal classifier 19 classifies the ST segment into one of the multiple predetermined categories using mapping data associating predetermined ranges of variance data values with corresponding categories of ST segment. The mapping data associates predetermined ranges of variance data values for populations of particular demographic characteristics including at least one of, age, weight, height and gender with corresponding categories of ST segment. The process of FIG. 1 terminates at step 931.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example, and is conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A graphical user interface (GUI), as used herein, comprises one or more display images, generated by a display processor and enabling user interaction with a processor or other device and associated data acquisition and processing functions.

The UI also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of FIGS. 2-13 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system derives and employs a set of parameters (Sign of ST Deviation, KLT features, Curve and line feature along with ΔJTon) to improve morphological classification of an ECG ST Segment (e.g., for Horizontal Depression and Downsloping Depression) and is also used in the classification of other morphologies. Further, the processes and applications may, in alternative embodiments, be located on one or more (e.g., distributed) processing devices on a network linking the units of FIG. 2. Any of the functions and steps provided in FIGS. 2-13 may be implemented in hardware, software or a combination of both.

What is claimed is:

1. A system for heart signal classification, comprising:
an interface for receiving an electrical signal waveform comprising an R-wave and including an ST segment portion associated with heart electrical activity of a patient over a heart beat cycle;
a signal processor for processing data representing said electrical signal waveform by
(a) fitting a curve to data representing said ST segment and
(b) applying a transform to the fitted curve to derive variance data indicating variance in the fitted curve; and
a signal classifier for classifying the ST segment into one of a plurality of predetermined categories associated with the fitted ST segment curve geometry in response to the derived variance data.

2. A system according to claim 1, wherein
said signal classifier classifies the ST segment into one of a plurality of predetermined categories associated with characteristics including (a) Concave Elevation and (b) Convex Elevation.

3. A system according to claim 2, wherein
said signal classifier classifies the ST segment into one of a plurality of predetermined categories associated with characteristics including (a) Upsloping Depression and (b) Horizontal Depression.

4. A system according to claim 3, wherein
said signal classifier classifies the ST segment into one of a plurality of predetermined categories associated with characteristics including Downsloping Depression.

5. A system according to claim 1, wherein
said transform comprises a KLT transform or another variance analysis transform.

6. A system according to claim 1, wherein
said transform performs Principal Component Analysis (PCA) to transform the data to a new coordinate system such that the greatest variance lies on a first coordinate called the first principal component.

7. A system according to claim 1, wherein
said signal processor adaptively fits a first degree curve or a second degree curve selected in response to a determined ST deviation value.

8. A system according to claim 1, wherein
said signal processor adaptively fits a curve or a line to an ST segment, selected in response to a determined ST deviation value indicating a positive or negative ST segment slope.

9. A system according to claim 1, wherein
said signal classifier classifies the ST segment into one of said plurality of predetermined categories using mapping data associating predetermined ranges of variance data values with corresponding categories of ST segment.

10. A system according to claim 9, wherein
said mapping data associates predetermined ranges of variance data values for populations of particular demographic characteristics including at least one of, age, weight, height and gender with corresponding categories of ST segment.

11. A system according to claim 1, wherein
said signal processor processes data representing said electrical signal waveform by
(a) identifying a J point in said electrical signal waveform,
(b) identifying a Ton point in said electrical signal waveform substantially occurring 80 milliseconds after said J point and
(c) determining a voltage difference between J point and Ton electrical signal waveform values; and
said signal classifier classifies the ST segment into one of a plurality of predetermined categories in response to the derived voltage difference value.

12. A system according to claim 11, wherein
said signal classifier classifies the ST segment into one of a plurality of predetermined categories associated with characteristics including (a) Horizontal Depression and (b) Downsloping Depression.

13. A system for heart signal classification, comprising:
an interface for receiving an electrical signal waveform comprising an R-wave and including an ST segment portion associated with heart electrical activity of a patient over a heart beat cycle;
a signal processor for processing data representing said electrical signal waveform by
(a) identifying a J point in said electrical signal waveform,
(b) identifying a Ton point in said electrical signal waveform substantially occurring 80 milliseconds after said J point and
(c) determining a voltage difference between J point and Ton electrical signal waveform values; and
a signal classifier for classifying the ST segment into one of a plurality of predetermined categories in response to the derived voltage difference value.

14. A system according to claim 13, wherein
said signal classifier classifies the ST segment into one of a plurality of predetermined categories associated with characteristics including (a) Horizontal Depression and (b) Downsloping Depression.

15. A system according to claim 13, wherein
said signal processor processes data representing said electrical signal waveform by
(a) fitting a curve to data representing said ST segment and
(b) applying a transform to the fitted curve to derive variance data indicating variance in the fitted curve; and
said signal classifier classifies the ST segment into one of a plurality of predetermined categories associated with fitted curve geometry in response to the derived variance data.

16. A method for heart signal classification, comprising the steps of:
receiving an electrical signal waveform comprising an R-wave and including an ST segment portion associated with heart electrical activity of a patient over a heart beat cycle;
processing data representing said electrical signal waveform by
(a) fitting a curve to data representing said ST segment and
(b) applying a transform to the fitted curve to derive variance data indicating variance in the fitted curve; and
classifying the ST segment into one of a plurality of predetermined categories associated with the fitted ST segment curve geometry in response to the derived variance data.
